(19) United States
(12) Patent Application Publication
(10) Pub. No.: US 2026/0036499 A1
(43) Pub. Date: Feb. 5, 2026

(54) VISIBLE TRANSPARENT SOIL MODEL TEST BOX WITH ADJUSTABLE SURROUNDING ROCK PRESSURE AND MANUFACTURING METHOD THEREFOR

(71) Applicant: Shijiazhuang Tiedao University, Shijiazhuang City (CN)

(72) Inventors: Zhichun LIU, Shijiazhuang City (CN); Fei WU, Shijiazhuang City (CN); Ranran QI, Shijiazhuang City (CN); Yunchao ZHOU, Shijiazhuang City (CN); Junmeng ZHANG, Shijiazhuang City (CN); Jianan GUO, Shijiazhuang City (CN); Guangshuo JIN, Shijiazhuang City (CN); Jingnan ZHAO, Shijiazhuang City (CN); Hao LI, Shijiazhuang City (CN)

(21) Appl. No.: 19/212,809

(22) Filed: May 20, 2025

(30) Foreign Application Priority Data
Jul. 31, 2024 (CN) 202411034497.9

(51) Int. Cl. G01N 3/04; G01N 3/16; G01N 33/24

(52) U.S. Cl. CPC G01N 3/04; G01N 3/16; G01N 33/24; G01N 2203/0019; G01N 2203/0037; G01N 2203/0423

(57) ABSTRACT

Provided are a visible transparent soil model test box with an adjustable surrounding rock pressure and a manufacturing method therefor. The visible transparent soil model test box includes pressure plates, force transferring plates, bolt assemblies and pressure boxes. The model box is assembled by the bolt assemblies, and then transparent soil is poured into the box. During the pouring of the transparent soil, the pressure boxes are buried inside the transparent soil and at inner sides of a left pressure plate, a right pressure plate and an upper pressure plate. After the transparent soil is consolidated, nuts are screwed to push the force transferring plates and the pressure plates by pressure gaskets to move inward to squeeze soil mass, and the pressure is controlled through readings of the pressure boxes, thus achieving automatic regulation of the surrounding rock pressure.

### CROSS-REFERENCE TO RELATED APPLICATION

This patent application claims the benefit and priority of Chinese Patent Application No. 202411034497.9, entitled “visible TRANSPARENT SOIL MODEL TEST BOX WITH ADJUSTABLE SURROUNDING ROCK PRESSURE AND MANUFACTURING METHOD THEREFOR” filed on Jul. 31, 2024, the disclosure of which is incorporated by reference herein in its entirety as part of the present application.

### TECHNICAL FIELD

The present disclosure relates to the technical field of deformation of surrounding rocks, and in particular to a visible transparent soil model test box with an adjustable surrounding rock pressure, and a manufacturing method therefor.

### BACKGROUND

With the development of tunnel in the direction of “long, large and deep burying”, the problem of squeezing surrounding rock becomes more and more prominent. Indoor test is one of the important means to study the deformation mechanism of squeezing surrounding rocks. In general, rock-soil mass like materials are configured by similar theory to simulate the original rock-soil mass. However, due to the lack of transparency of conventional materials, it is difficult to completely observe strain state of the surrounding rock after tunnel excavation, which has brought certain obstacles to scientific research. In recent years, due to the rapid development of transparent soil technology, the visualization of internal deformation of soil mass has been implemented. However, if the test is still carried out by using the traditional steel plate model test box, it is still impossible to observe the internal displacement of the soil mass, and the existing transparent soil model test box cannot achieve the independent adjustment of surrounding rock pressure. Therefore, it is necessary to design a visible transparent soil model test box with an adjustable surrounding rock pressure.

### SUMMARY

The embodiments aim to provide a visible transparent soil model test box with an adjustable surrounding rock pressure and a manufacturing method therefor to solve the problems in the prior art, which can be used to study the change rule of displacement and strain of a surrounding rock with excavation step length of the tunnel, sealing distance and other factors, and can achieve the adjustment of greater surrounding rock pressure by changing the thicknesses of a left pressure plate, a right pressure plate and an upper pressure plate, thus further revealing the deformation mechanism of deep-buried squeezing surrounding rock.

To achieve the objective above, the present disclosure provides the following solutions. The present disclosure provides a visible transparent soil model test box with an adjustable surrounding rock pressure, including pressure plates, force transferring plates, bolt assemblies and pressure boxes. The pressure plates include an upper pressure plate, a left pressure plate and a right pressure plate. The force transferring plates include an upper force transferring plate, a lower force transferring plate, a left force transferring plate, and a right force transferring plate. The lower force transferring plate is placed on a backing plate, the left force transferring plate and the right force transferring plate are connected by the some of bolt assemblies, the upper force transferring plate and the lower force transferring plate are connected by others of the bolt assemblies, and both ends of the left force transferring plate and the right force transferring plate are respectively provided with a front tunnel cross-sectional plate and a rear tunnel cross-sectional plate. The upper force transferring plate, the lower force transferring plate, the left force transferring plate, the right force transferring plate, the front tunnel cross-sectional plate and the rear tunnel cross-sectional plate form a model test box. The upper pressure plate, the left pressure plate and the right pressure plate are respectively arranged at inner sides of the upper force transferring plate, the left force transferring plate and the right force transferring plate. Transparent soil is placed in the model test box. One of the pressure boxes is arranged in the transparent soil, and others of the pressure boxes are respectively arranged between the transparent soil and the upper pressure plate, between the transparent soil and the left pressure plate, and between the transparent soil and the right pressure plate.

In some embodiments, each of the bolt assemblies includes a threaded rod, a nut, and a pressure gasket. The left force transferring plate and the right force transferring plate are connected by four transverse threaded rods, and the four transverse threaded rods are respectively arranged adjacent to four corners of the left force transferring plate and the right force transferring plate. Both end portions of each of the four transverse threaded rods respectively extend out of the left force transferring plate and the right force transferring plate and are tightened or unscrewed by nuts, and the pressure gasket is arranged between an each of the both end portions of each of the four transverse threaded rods and the left force transferring plate or the right force transferring plate. The upper force transferring plate and the lower force transferring plate are connected by four vertical threaded rods, and the four vertical threaded rods are respectively arranged adjacent to four corners of the upper force transferring plate and the lower force transferring plate. Both end portions of each of the four vertical threaded rods respectively extend out of the upper force transferring plate and the lower force transferring plate and are tightened or unscrewed by nuts, respectively, and the pressure gasket is arranged between each of the both end portions of each of the four vertical threaded rods and the upper force transferring plate or the lower force transferring plate.

In some embodiments, the visible transparent soil model test box with an adjustable surrounding rock pressure further includes four rectangular steel pipes. The four rectangular steel pipes are configured to respectively abut against and support both ends of the front tunnel cross-sectional plate and both ends of the rear tunnel cross-sectional plate. Both ends of each of the four transverse threaded rods respectively penetrates through corresponding ones of the four rectangular steel pipes and then extends out from the left force transferring plate and the right force transferring plate.

The present disclosure further provides a manufacturing method for a visible transparent soil model test box with an adjustable surrounding rock pressure. The manufacturing method is applied to the visible transparent soil model test box with an adjustable surrounding rock pressure above, and includes:
placing the lower force transferring plate on the backing plate, and connecting the left force transferring plate and the right force transferring plate by threaded rods, rectangular steel pipes, pressure gaskets and nuts; respectively inserting the front tunnel cross-sectional plate and the rear tunnel cross-sectional plate into inner front and rear sides of the left force transferring plate and inner front and rear sides of the right force transferring plate; tightening the nuts to make the left force transferring plate and the right force transferring plate clamp the front tunnel cross-sectional plate and the rear tunnel cross-sectional plate to keep a device stable; respectively inserting the left pressure plate and the right pressure plate into an inner side of the left force transferring plate and an inner side of the right force transferring plate; and unscrewing the nuts at end portions of transverse threaded rods of the threaded rods after the left pressure plate and the right pressure plate are inserted, thus completing assembling of the model test box; and
burying the pressure boxes in soil mass and at an inner side of the left pressure plate, an inner side of the right pressure plate and an upper side of transparent soil while pouring the transparent soil into the model test box; and after the transparent soil is consolidated, placing the upper pressure plate above the transparent soil and placing the upper force transferring plate above the upper pressure plate, and connecting the upper force transferring plate and the lower force transferring plate by vertical threaded rods of the threaded rods, the nuts and the pressure gaskets, adjusting a distance for squeezing the soil mass by rotating the nuts, and observing readings of the pressure boxes to control a degree of rotating the nuts, thus achieving controllable adjustment of a surrounding rock pressure.

Compared with the prior art, the embodiments have the following technical effects.

The present disclosure provides the visible transparent soil model test box with an adjustable surrounding rock pressure and the manufacturing method therefor. The visible transparent soil model test box includes pressure plates, force transferring plates, bolt assemblies and pressure boxes. The model box is assembled by the bolt assemblies, and then transparent soil is poured into the box. During the pouring of the transparent soil, the pressure boxes are buried inside the transparent soil and at inner sides of the left pressure plate, the right pressure plate and the upper pressure plate. After the transparent soil is consolidated, the nuts are screwed to push the force transferring plates and the pressure plates by the pressure gaskets to move inward to squeeze soil mass, and the pressure is controlled through readings of the pressure boxes, thus achieving automatic regulation of the surrounding rock pressure. The device can adjust a support reaction force conveniently, is safe, reliable, low in cost and has a good application prospect.

### BRIEF DESCRIPTION OF THE DRAWINGS

To describe the technical solutions of the embodiments of the present disclosure or in the prior art more clearly, the following briefly introduces the drawings required for describing the embodiments. Apparently, the drawings in the following description show merely some embodiments of the present disclosure, and those of ordinary skill in the art may still derive other drawings from these drawings without creative efforts.

FIG. 1 is a three-dimensional diagram of a visible transparent soil model test box with an adjustable surrounding rock pressure;

FIG. 2 is a schematic diagram of an internal structure of a model text box after an upper force transferring plate is removed;

FIG. 3 is a schematic structural diagram of a left pressure plate and a right pressure plate;

FIG. 4 is a schematic structural diagram of a left force transferring plate and a right force transferring plate;

FIG. 5 is a schematic structural diagram of a tunnel cross-sectional plate;

FIG. 6 is a schematic structural diagram of an upper pressure plate;

FIG. 7 is a schematic structural diagram of an upper force transferring plate and a lower force transferring plate;

FIG. 8 is a schematic structural diagram of a rectangular steel pipe.

List of the reference characters: 1 upper pressure plate; 2 right pressure plate; 3 right force transferring plate; 4 front tunnel cross-sectional plate; 5 lower force transferring plate; 6-nut; 7 threaded rod; 8 rectangular steel pipe; 9 pressure gasket; 10 transparent soil; and 11 pressure box.

### DETAILED DESCRIPTION OF THE EMBODIMENTS

The following clearly and completely describes the technical solutions in the embodiments of the present disclosure with reference to the drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

The embodiments aim to provide a visible transparent soil model test box with an adjustable surrounding rock pressure and a manufacturing method therefor to solve the problems in the prior art, which can be used to study the change rule of displacement and strain of a surrounding rock with excavation step length of the tunnel, sealing distance and other factors, and can achieve the adjustment of greater surrounding rock pressure by changing the thicknesses of a left pressure plate, a right pressure plate and an upper pressure plate, thus further revealing the deformation mechanism of deep-buried squeezing surrounding rock.

In order to make the objectives, features and advantages of the present disclosure more clearly, the present disclosure is further described in detail below with reference to the drawings and specific embodiments.

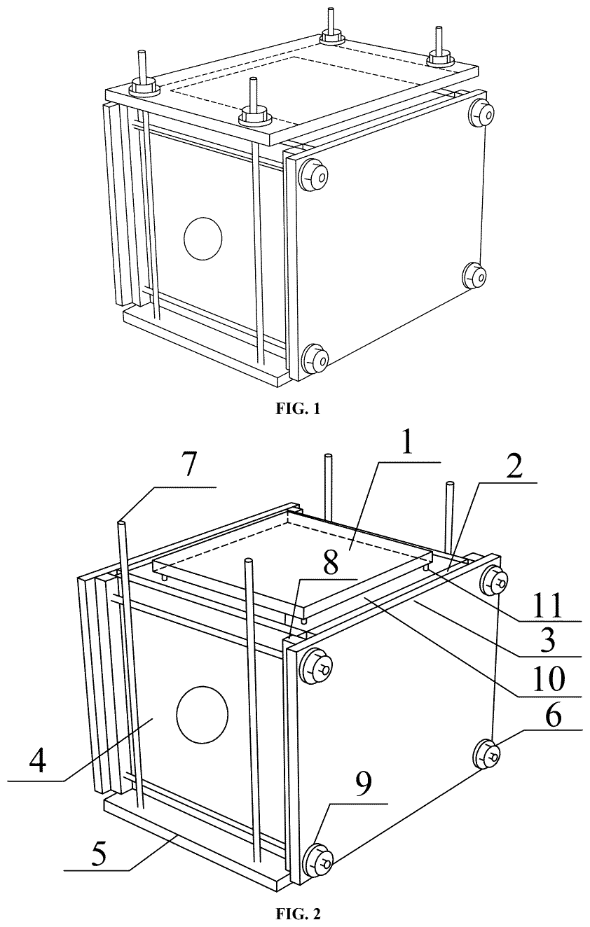

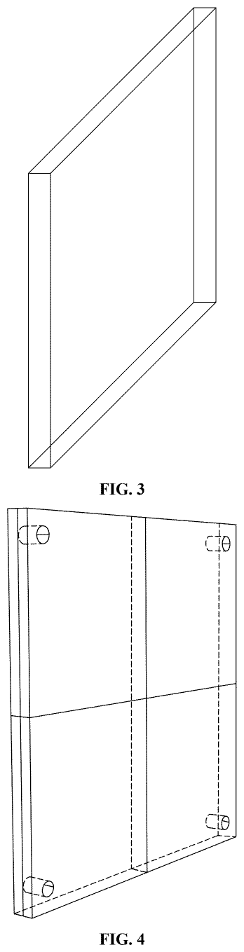

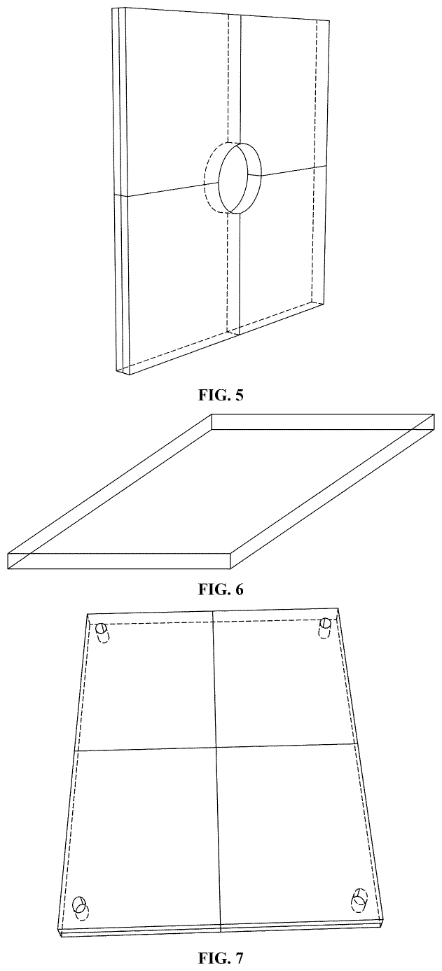

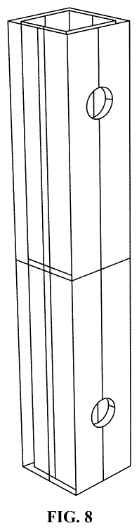

As shown in FIG. 1 to FIG. 8, the present disclosure provides the visible transparent soil model test box with an adjustable surrounding rock pressure, including pressure plates, force transferring plates, bolt assemblies and pressure boxes 11. The pressure plates include an upper pressure plate 1, a left pressure plate and a right pressure plate 2. The force transferring plates include an upper force transferring plate, a lower force transferring plate 5, a left force transferring plate and a right force transferring plate 3. The lower force transferring plate 5 is placed on a backing plate, the left force transferring plate and the right force transferring plate 3 are connected by the bolt assemblies, the upper force transferring plate and the lower force transferring plate 5 are connected by the bolt assemblies, and both ends of the left force transferring plate and the right force transferring plate 3 are respectively provided with a front tunnel cross-sectional plate 4 and a rear tunnel cross-sectional plate. The upper force transferring plate, the lower force transferring plate 5, the left force transferring plate, the right force transferring plate 3, the front tunnel cross-sectional plate 4 and the rear tunnel cross-sectional plate form a model test box. The upper pressure plate 1, the left pressure plate and the right pressure plate 2 are respectively arranged at inner sides of the upper force transferring plate, the left force transferring plate and the right force transferring plate 3. Transparent soil 10 is placed in the model test box, and the pressure boxes 11 are respectively arranged between the transparent soil 10 and the upper pressure plate 1, between the transparent soil 10 and the left pressure plate, and between the transparent soil 10 and the right pressure plate 2.

In one of embodiments, each bolt assembly includes a threaded rod 7, a nut 6 and a pressure gasket 9. The left force transferring plate and the right force transferring plate 3 are connected by four transverse threaded rods 7, and the four transverse threaded rods 7 are respectively arranged adjacent to four corners of the left force transferring plate and the right force transferring plate 3. Both ends of each transverse threaded rod 7 respectively extend out of the left force transferring plate and the right force transferring plate 3 and are tightened or unscrewed by the nuts 6, and the pressure gasket 9 is arranged between an end portion of the transverse threaded rod 7 and the left force transferring plate or the right force transferring plate 3. The upper force transferring plate and the lower force transferring plate 5 are connected by four vertical threaded rods 7, and the four vertical threaded rods 7 are respectively arranged adjacent to four corners of the upper force transferring plate and the lower force transferring plate 5. Both ends of each vertical threaded rod 7 respectively extend out of the upper force transferring plate and the lower force transferring plate 5 and are tightened or unscrewed by the nuts 6, respectively, and the pressure gasket 9 is arranged between an end portion of the vertical threaded rod 7 and the upper force transferring plate or the lower force transferring plate 5.

In one of embodiments, the visible transparent soil model test box with an adjustable surrounding rock pressure further includes four rectangular steel pipes 8. The four rectangular steel pipes 8 are configured to respectively abut against and support both ends of the front tunnel cross-sectional plate 4 and both ends of the rear tunnel cross-sectional plate. Both ends of the transverse threaded rod 7 respectively penetrate through the rectangular steel pipes 8 and then extend out from the left force transferring plate and the right force transferring plate 3.

In one of embodiments, the pressure box 11 is further arranged in the transparent soil 10.

### Embodiment 1

The following is a description of the relationship.

After the device is installed, it is a model test box with an internal space of 60 cm×60 cm×60 cm. The upper pressure plate 1 is transparent tempered glass of 50 cm×50 cm×3 cm, and the thickness of 3 cm is an upper distance for compressing the soil mass. A deformation of 5 cm is allowed on each side of the upper pressure plate 1 for simultaneous compression and movement of the left pressure plate and the right pressure plate while achieving downward movement of the upper pressure plate 1. If the upper pressure plate 1 is made into 60 cm×60 cm×60 cm, it is impossible to achieve simultaneous pressurization on the upper side, the left side and the right side. When the pressure is applied to the upper pressure plate 1, part of the soil will be squeezed out from a gap. In practice, it was found during the test that the squeezed soil can be basically ignored, this is because the gap is gradually reduced with the pressurization, and no soil in the gap was found to be squeezed out during the test. In view of the size of the model box, the internal size is 60 cm×60 cm×60 cm, a hole diameter is 8 cm, and a distance from an edge of the hole to any side is 26 cm, which is more than 3 times the hole diameter. According to the theory of elasticity, the influence of this boundary on the internal state change of soil mass can be ignored, and thus the test results will not be affected. Both the left pressure plate and the right pressure plate 2 are transparent tempered glass of 60 cm×60 cm×3 cm, and the thickness of 3 cm is the distance for compressing the soil mass on the left side and the right side. Both the left force transferring plate and the right force transferring plate 3 are transparent tempered glass of 80 cm×60 cm×3 cm, holes with a diameter of 2 cm are formed in four corners of the left force transferring plate and the right force transferring plate 3, and a distance from the hole circle to the edge is 5 cm. Both the front tunnel cross-section plate 4 and the rear tunnel cross-section plate are transparent tempered glass of 61 cm×60 cm×3 cm, and a hole with a diameter of 8 cm is formed in the center to simulate the tunnel. The front tunnel cross-section plate 4 and the rear tunnel cross-section plate can be replaced according to the tunnel cross section studied in detail. Both the upper force transferring plate and the lower force transferring plate 5 are transparent tempered glass of 84 cm×61 cm×3 cm, holes with a diameter of 4 cm are formed in four corners of the upper force transferring plate and the lower force transferring plate 5, a distance from the center of the hole to a short side is 4 cm, and a distance from the center of the hole to a long side is 10 cm.

The manufacturing method for the visible transparent soil model test box with an adjustable surrounding rock pressure is as follows.

The lower force transferring plate 5 is placed on the backing plate, and the left force transferring plate and the right force transferring plate 3 are connected by the threaded rods 7, the rectangular steel pipes 8, the pressure gaskets 9 and the nuts 6, and then the front tunnel cross-sectional plate 4 and the rear tunnel cross-sectional plate are respectively inserted into front and rear sides of the left force transferring plate and the right force transferring plate, and the nuts 6 are tightened to make the left force transferring plate and the right force transferring plate 3 clamp the front tunnel cross-sectional plate 4 and the rear tunnel cross-sectional plate to keep a device stable. The left pressure plate and the right pressure plate 2 are respectively inserted into the left side and the right side. After being inserted into the left pressure plate and the right pressure plate 2, the nuts 6 at end portions of the transverse threaded rods 7 are unscrewed to make the left pressure plate and the right pressure plate 3 move outward by a distance of ⅔ of the thickness of the pressure plate, and the distance of ⅔ of the thickness of the pressure plate is the distance that the soil mass on the left side and the right side can be compressed. At this time, assembling of the model test box is completed. When the transparent soil 10 is poured into the model test box, the pressure boxes 11 should be buried in the soil mass and at the inner side of the left pressure plate and the inner side the right pressure plate and the upper side of the transparent soil 10. After the transparent soil 10 is consolidated, the upper pressure plate 1 is placed on the transparent soil 10, and a thickness of the upper pressure plate 1 is the distance that the soil mass at the upper side can be compressed. Then, the upper force transferring plate (the upper and lower force transferring plates have the same size) is placed on the upper pressure plate 1. The upper force transferring plate and the lower force transferring plate are connected by the vertical threaded rods 7, the nuts 6 and the pressure gaskets 9, the distance for squeezing the soil mass is adjusted by rotating the nuts 6, the degree of rotating the nuts 6 is controlled by observing readings of the pressure boxes 11, thus achieving controllable adjustment of the surrounding rock pressure. The scheme adopted during the test is that the nuts 6 are diagonally rotated by two people at the same time, and the readings of the pressure boxes 11 are observed. It is not that one person only rotates one nut 6 at a time, and the transfer of applied pressure takes time, and the pressure cannot be transferred to the center of the model box immediately after the boundary pressure is applied, and in general, it takes several hours after the boundary pressure is applied, and then the tunnel can be excavated, so there is completely time to control the pressure of the side plate by adjusting the nuts 6. The nut 6 is rotated with a wrench, which belongs to a conventional device in the prior art. If electromechanical measures are used, the applied pressure will be unstable and is difficult to control. It is possible that at the moment when the switch is turned on or off, the side plate has already produced a great amount of compression, which is difficult to control. Finally, the wrench is used to achieve the expected effect.

It should be noted that it is apparent to those skilled in the art that the present disclosure is not limited to the details of the above exemplary embodiments, and can be implemented in other specific forms without departing from the spirit or basic characteristics of the present disclosure. Therefore, the embodiments should be considered as exemplary and non-limiting in all aspects, and the scope of the present disclosure is defined by the appended claims rather than the above description, so it is intended to embrace all changes that fall within the meaning and range of equivalents of the claims, and any reference signs in the claims should not be regarded as limiting the claims involved.

Specific examples are used herein for illustration of the principles and embodiments of the present disclosure. The description of the embodiments is merely used to help illustrate the method and its core principles of the present disclosure. In addition, those of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. A visible transparent soil model test box with an adjustable surrounding rock pressure, comprising:
pressure plates, comprising an upper pressure plate, a left pressure plate and a right pressure plate;
force transferring plates, comprising an upper force transferring plate, a lower force transferring plate, a left force transferring plate and a right force transferring plate; wherein the lower force transferring plate is placed on a backing plate; both ends of the left force transferring plate and the right force transferring plate are respectively provided with a front tunnel cross-sectional plate and a rear tunnel cross-sectional plate; the upper force transferring plate, the lower force transferring plate, the left force transferring plate, the right force transferring plate, the front tunnel cross-sectional plate and the rear tunnel cross-sectional plate form a model test box;
bolt assemblies, wherein the left force transferring plate and the right force transferring plate are connected by some of the bolt assemblies, the upper force transferring plate and the lower force transferring plate are connected by others of the bolt assemblies; and
pressure boxes,
wherein the upper pressure plate, the left pressure plate and the right pressure plate are respectively arranged at inner sides of the upper force transferring plate, the left force transferring plate and the right force transferring plate;
wherein transparent soil is placed in the model test box, one of the pressure boxes is arranged in the transparent soil, and others of the pressure boxes are respectively arranged between the transparent soil and the upper pressure plate, between the transparent soil and the left pressure plate, and between the transparent soil and the right pressure plate.

2. The visible transparent soil model test box with an adjustable surrounding rock pressure according to claim 1, wherein each of the bolt assemblies comprises a threaded rod, a nut and a pressure gasket;
the left force transferring plate and the right force transferring plate are connected by four transverse threaded rods, and the four transverse threaded rods are respectively arranged adjacent to four corners of the left force transferring plate and the right force transferring plate; both end portions of each of the four transverse threaded rods respectively extend out of the left force transferring plate and the right force transferring plate and are tightened or unscrewed by nuts, and the pressure gasket is arranged between each of the both end portions of each of the four transverse threaded rods and the left force transferring plate or the right force transferring plate;
the upper force transferring plate and the lower force transferring plate are connected by four vertical threaded rods, and the four vertical threaded rods are respectively arranged adjacent to four corners of the upper force transferring plate and the lower force transferring plate; and
both end portions of each of the four vertical threaded rods respectively extend out of the upper force transferring plate and the lower force transferring plate and are tightened or unscrewed by nuts, and the pressure gasket is arranged between each of the both end portions of each of the four vertical threaded rods and the upper force transferring plate or the lower force transferring plate.

3. The visible transparent soil model test box with an adjustable surrounding rock pressure according to claim 2, further comprising four rectangular steel pipes configured to respectively abut against and support both ends of the front tunnel cross-sectional plate and both ends of the rear tunnel cross-sectional plate, and both ends of each of the four transverse threaded rods respectively penetrates through corresponding ones of the four rectangular steel pipes and then extends out from the left force transferring plate and the right force transferring plate.

4. A manufacturing method for a visible transparent soil model test box with an adjustable surrounding rock pressure, wherein the manufacturing method is applied to the visible transparent soil model test box with an adjustable surrounding rock pressure according to claim 1 and comprises:
placing the lower force transferring plate on the backing plate, and connecting the left force transferring plate and the right force transferring plate by threaded rods, rectangular steel pipes, pressure gaskets and nuts; respectively inserting the front tunnel cross-sectional plate and the rear tunnel cross-sectional plate into inner front and rear sides of the left force transferring plate and inner front and rear sides of the right force transferring plate; tightening the nuts to make the left force transferring plate and the right force transferring plate clamp the front tunnel cross-sectional plate and the rear tunnel cross-sectional plate to keep a device stable; respectively inserting the left pressure plate and the right pressure plate into an inner side of the left force transferring plate and an inner side of the right force transferring plate; and unscrewing the nuts at end portions of transverse threaded rods of the threaded rods after the left pressure plate and the right pressure plate are inserted, thus completing assembling of the model test box; and
burying the pressure boxes in soil mass and at an inner side of the left pressure plate, an inner side of the right pressure plate and an upper side of transparent soil while pouring the transparent soil into the model test box; and after the transparent soil is consolidated, placing the upper pressure plate above the transparent soil and placing the upper force transferring plate above the upper pressure plate, and connecting the upper force transferring plate and the lower force transferring plate by vertical threaded rods of the threaded rods, the nuts and the pressure gaskets, adjusting a distance for squeezing the soil mass by rotating the nuts, and observing readings of the pressure boxes to control a degree of rotating the nuts, thus achieving controllable adjustment of a surrounding rock pressure.

5. The manufacturing method for a visible transparent soil model test box with an adjustable surrounding rock pressure according to claim 4, wherein each of the bolt assemblies comprises a threaded rod, a nut and a pressure gasket; the left force transferring plate and the right force transferring plate are connected by four transverse threaded rods, and the four transverse threaded rods are respectively arranged adjacent to four corners of the left force transferring plate and the right force transferring plate; both end portions of each of the four transverse threaded rods respectively extend out of the left force transferring plate and the right force transferring plate and are tightened or unscrewed by nuts, and the pressure gasket is arranged between each of the both end portions of each of the four transverse threaded rods and the left force transferring plate or the right force transferring plate; the upper force transferring plate and the lower force transferring plate are connected by four vertical threaded rods, and the four vertical threaded rods are respectively arranged adjacent to four corners of the upper force transferring plate and the lower force transferring plate; and both end portions of each of the four vertical threaded rods respectively extend out of the upper force transferring plate and the lower force transferring plate and are tightened or unscrewed by nuts, and the pressure gasket is arranged between each of the both end portions of each of the four vertical threaded rods and the upper force transferring plate or the lower force transferring plate.

6. The manufacturing method for a visible transparent soil model test box with an adjustable surrounding rock pressure according to claim 5, wherein the visible transparent soil model test box with an adjustable surrounding rock pressure further comprises four rectangular steel pipes, the four rectangular steel pipes are configured to respectively abut against and support both ends of the front tunnel cross-sectional plate and both ends of the rear tunnel cross-sectional plate; and both ends of each of the four transverse threaded rods respectively penetrates through corresponding ones of the four rectangular steel pipes and then extends out from the left force transferring plate and the right force transferring plate.
